(19) United States
(12) Patent Application Publication
(10) Pub. No.: US 2024/0050268 A1
(43) Pub. Date: Feb. 15, 2024

(54) Tongue Training Appliance

(71) Applicant: Janine Cline, New York, NY (US)

(72) Inventor: Janine Cline, New York, NY (US)

(21) Appl. No.: 18/445,189

(22) Filed: May 17, 2023

Related U.S. Application Data
(63) Continuation-in-part of application No. 17/534,834, filed on Nov. 24, 2021, now Pat. No. 11,712,365.

(51) Int. Cl. A61F 5/58; A61F 5/50; A61F 5/56

(52) U.S. Cl. CPC A61F 5/58; A61F 5/50; A61F 5/566

(57) ABSTRACT

A custom fit tongue training appliance for training the tongue of a patient with a speech disorder or orthodontic conditions is disclosed. The appliance comprises a shell which covers the upper teeth having a labial side and a lingual side, a platform, an aperture, and protrusions. The appliance provides a tactile cue to train the tongue for proper lingual resting posture that promotes beneficial myofunctional habits and improves speech quality.

### CROSS-REFERENCE TO RELATED APPLICATIONS/CLAIM OF PRIORITY

This application is a continuation-in-part of U.S. application Ser. No. 17/534,834, filed Nov. 24, 2021, the contents of which are incorporated by reference herein in their entirety.

### TECHNICAL FIELD

The present application relates to speech and orthodontic therapy devices.

### BACKGROUND

This invention is directed to a tongue training appliance and, more particularly, to an appliance for eliminating tongue thrust against the upper teeth and for habituating proper placement of the tongue for speech, swallowing and resting posture.

When the tongue is in its natural resting position, the tip positions on the incisal papilla at the anterior part of the upper palate. The tongue exerts pressure on the teeth when its rest position is misplaced and due to its movement while swallowing. The tongue then thrusts between 500-1000 times a day during swallowing. This can place pressure on the teeth resulting in dental malocclusions and speech problems. If left untreated, tongue thrusts can affect overall speech intelligibility which results in distortions of lingual alveolar and lingual palatal sounds or misplaced production of those sounds. These distortions occur secondary to their tongue thrust and result in an inability to manage saliva when speaking, weakness in the lingual musculature and poor tongue/jaw dissociation. Tongue thrusts can also lead to malocclusion, poor oral hygiene, and symptoms associated with temporomandibular joint disorder (TMJ/D).

Proper tongue placement is necessary for the production of speech sounds, and the present tongue training appliance improves speech intelligibility. The tongue training appliance strengthens the orofacial muscles, while promoting the correct resting posture of the tongue and eliminates noxious oral habits including thumb, finger and tongue sucking and nail biting. In the present specification, “noxious oral habits” includes one or more posture or habits that are not correct and include incorrect tongue position, tongue thrusting, digit sucking, an incorrect swallowing pattern and mouth breathing. The appliance encourages proper resting posture of the tongue and lips, which maximizes airway space for breathing. The appliance improves nasal breathing by maximizing and conditioning habitual breathing through the nose by facilitating a closed mouth posture and the filtration of air through the nasal cavity.

Myofunctional therapy supports neuromuscular re-education of the tongue while at rest, swallowing and speaking. However, retraining of the tongue to identify the correct palatal position or “spot” using myofunctional therapy, while an important adjunct, requires considerable patient compliance. It is recommended that the patient practice this exercise up to 60 minutes each day. For children, who frequently have short attention spans, compliance relies heavily upon parental supervision and it is difficult to ensure the exercise is being carried out correctly. The appliance may be used together with a myofunctional therapy program.

One device that interferes with the thrust of the tongue at rest, when swallowing, and during other non-nutritive sucking habits is a tongue crib. Tongue cribs cover the incisal papilla and the alveolar ridge. Therefore proper retraining of the tongue at rest, when swallowing, and when speaking is unable to take place. A metal strainer traverses the palate and interferes with the tongue passive movements of speech, swallowing, non-nutritive sucking habits, and the resting posture of the tongue on the upper dentition. Tongue cribs are often found to be too intrusive and without myofunctional therapy the patient does not know where there tongue should rest. Therefore, the patient feels like they are choking. The appliance is not esthetic and is visible when the patient smiles.

Incorrect tongue position also occurs when a patient engages in non-nutritive oral habits including, but not limited to digit sucking, tongue sucking, nail biting, bruxism, and tongue thrust. A habit is an action or condition which by repetition has become subconscious. Noxious oral habits can lead to a number of conditions that are treated with an orthodontic approach such as braces and/or surgery. Braces are expensive and have disadvantages such as tooth decay or gum disease, allergic reactions, soft tissue injuries and canker sores, root resorption, decalcification and cavities, and failure or relapse. Surgery is both expensive and invasive. Dental malocclusions are treated with functional devices such as the Frankel appliance or surgery. While functional devices may correct a malocclusion, unlike the present appliance, they do not retrain the tongue for proper oral posture and do not support neuromuscular re-education.

The thickness of other current plastic appliances interferes with the ability to occlude completely and prohibits the usage of the masseter muscles. The inability to completely occlude interferes with the engagement of the masseters, which negatively impacts chewing and swallowing. Underdeveloped masseter muscles also interfere with the body's overall alignment. The masseter muscles are designed to hold a patient's head back and in alignment with their body. If a patient is unable to occlude completely a space is created which will allow the tongue space to thrust, therefore perpetuating a non-nutritive tongue thrust habit. These previous appliances promote an anterior tongue placement and the reliance on compensatory strategies for swallowing, which can have an adverse effect on an individual's overall occlusion.

The tongue training appliance described herein overcomes the above-mentioned disadvantages and meets the need for new alternative appliances and treatment in the fields of speech therapy, orthodontics and myofunctional therapy with improved patient outcomes.

### SUMMARY

A first aspect relates to a tongue training appliance which is custom fitted to the patient's maxillary front teeth having:

a shell or retainer which fits securely over the maxillary teeth having an anterior labial side and a posterior lingual side;

a plurality of protrusions on the posterior lingual side of the shell;

a platform extending from the posterior lingual side of the shell; and

an aperture located at the end of central incisors at the anterior part of an upper palate where the tongue should rest.

The appliance shell or retainer fits securely over the upper or maxillary teeth having a labial side and a lingual side. The labial side faces the lips and the lingual side faces the tongue.

On the lingual side are protrusions and a hole or aperture (“spot”) located at the end of the incisor papilla at the anterior part of the upper palate where the tongue should rest.

The appliance fits over the maxillary front six teeth, i.e., the two central incisors, two lateral incisors, and two cuspid teeth, although the extent of teeth coverage varies to include as few as four and as many as all the maxillary teeth, if desired. In one aspect, the appliance fits over all the maxillary teeth including the molars.

In one aspect, where the appliance fits over all the maxillary teeth including the molars, the surface of the molars is exposed.

The protrusions irritate the tongue tip (palate rugae) when they come into contact with the tongue. When the tongue extends beyond its proper position, the tongue contacts the protrusions, and is irritated. The discomfort causes withdrawal of the tongue away from the protrusions and toward its proper position.

In one aspect, the appliance has rows of protrusions on each tooth. In other aspects, the appliance does not have any protrusions.

In one aspect, the protrusions on the posterior lingual side of the shell are on the posterior lingual side of the maxillary four front teeth, the central and lateral incisors. In another aspect, the protrusions are on the posterior lingual side of the shell maxillary eight front teeth, the central incisors, lateral incisors, bilateral cuspids, and bilateral first premolars.

In one aspect, the protrusions on the posterior lingual side of the shell are a plurality of spikes or pins, or oblong or without a pointed end.

Another aspect relates to the appliance as esthetic and not readily visible.

Another aspect of the appliance is that it is monolithic (one piece) and manufactured using 3-D printing.

Another aspect of the appliance is that it is thin and made from soft resin material.

The appliance corrects and habituates proper lingual resting posture and improves overall speech intelligibility. Optionally, in any of the preceding aspects, there is a method of eliminating noxious oral habits, improving nasal breathing, maximizing airway space to support sleep disordered breathing, improving overall occlusion and reducing symptoms associated with temporomandibular joint problems.

Yet another aspect pertains to an appliance having a thin shell which permits the patient to speak and eat while the appliance is being worn.

The custom fit manufacturing process involves: scanning a patient's teeth with an intra-oral scanner, using a CAD/CAM software, using resin with a 3-D printer and curing the produced appliance.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background.

### BRIEF DESCRIPTION OF THE DRAWINGS

The present disclosure is directed to an appliance for tongue training to assume its proper position at rest and during swallowing. Aspects of the present disclosure are illustrated by way of example and are not limited by the accompanying figures for which like references indicate like elements.

FIG. 1 is an orientation drawing showing where the appliance is placed on the maxillary teeth.

FIG. 2 shows a front perspective view of one embodiment of the tongue training appliance having a custom fit shell covering the maxillary front six teeth.

FIG. 3 shows a front perspective view of another embodiment of the tongue training appliance having a custom fitted shell covering all the maxillary teeth bilaterally with the shell having only a lingual side for the front six teeth and having the surface of the molars exposed.

FIG. 4 shows a front perspective view of another embodiment of the tongue training appliance having a custom fitted shell covering all the maxillary teeth bilaterally.

FIG. 5 shows a front perspective view of another embodiment of the tongue training appliance having a custom fitted shell covering the maxillary front eight teeth, all having a plurality of protrusions.

### DETAILED DESCRIPTION

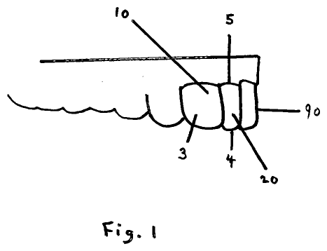

FIG. 1 depicts the appliance (3) with a shell (4) that extends to the gum line (5), and rests between the lips (not shown) and the maxillary anterior teeth (10) having a labial side (20) on the labial teeth surface and a lingual side (25) (not shown) on the lingual teeth surface on the opposite side.

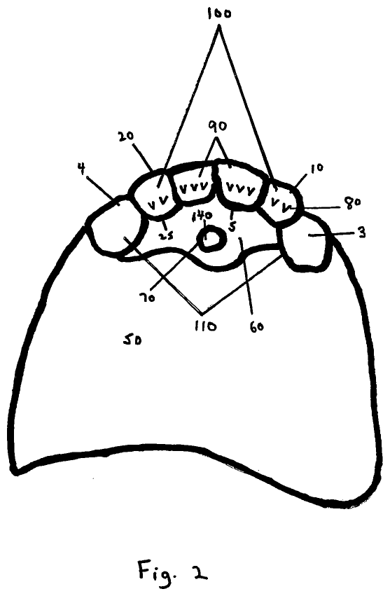

Turning now to the specific embodiments of the invention, as shown in the FIG. 2, each appliance (3) fits on the anterior teeth (10). The anterior teeth (10) are the two central incisors (90), two lateral incisors (100) and bilateral cuspids (110). Thus, the shell fits over the six anterior teeth (10) and is custom manufactured to the shape of the teeth and bite. The shell (4) fits securely over the anterior teeth (10) having a labial side (20) and a lingual side (25) and extends down to the gum line (5) and having a plurality of protrusions (80) extending perpendicularly from the posterior lingual side of the shell (25). A platform (60) extends from a posterior lingual side of the shell (25), and specifically from the gum line (5) of the six anterior teeth (10). The platform contains a round aperture (70) located at the end of the central incisor papilla (130) (not shown) at the anterior part of the upper palate (50) where the tongue (120) (not shown) should rest, also known as the “spot” (140).

In one aspect, the appliance (3) is similar to that of FIG. 2 but the main difference is that it does not contain protrusions.

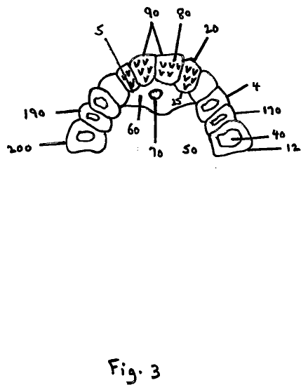

In one aspect, shown in FIG. 3, the shell (4) fits over all maxillary teeth (170), both anterior (10) and posterior (12) but the occlusal surface (40) of the posterior teeth (12) is exposed, and the shell (4) portion covering the central incisors (90) and the lateral incisors (100) has a posterior lingual side only (25) but no anterior labial side (20). The anterior lingual side may be with protrusions (80) or without protrusions (80). A platform (60) extends from the posterior lingual side of the shell (25), and specifically from the gum line (5) of the six anterior teeth (10). The platform contains a round aperture (70) located at the end of the central incisor papilla (130) (not shown) at the anterior part of the upper palate (50) where the tongue (120) (not shown) should rest, also known as the “spot” to those skilled in the art (140) (not shown). The occlusal surface (40) is the surface that aids in chewing with the tooth. The surface of the posterior teeth which include pre-molars (190) and molars (200) are exposed to allow for masseter engagement and to allow for the anterior teeth (10) to meet when biting.

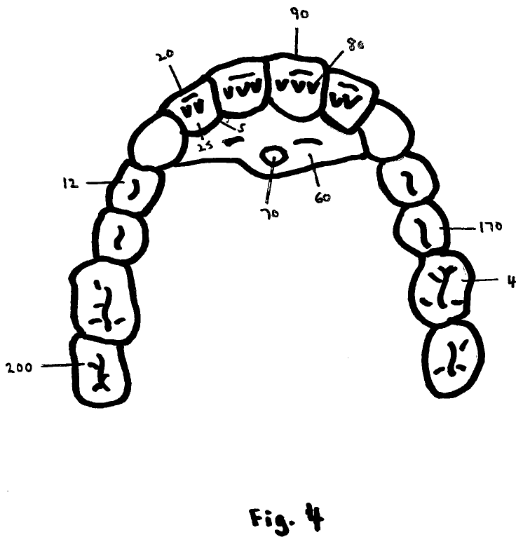

In one aspect, shown in FIG. 4, the shell (4) fits over all maxillary teeth (170), including molars (200), both anterior (10) and posterior (12), having a labial side (20) and a lingual side (25) and extends down to the gum line (5) and having a plurality of protrusions (80) extending perpendicularly from the posterior lingual side of the shell (25). A platform (60) extends from posterior lingual side of the shell (25), and specifically from the gum line (5) of the six anterior teeth (10). The platform contains a round aperture (70) at the anterior part of the upper palate (50) where the tongue (120) (not shown) should rest, also known as the “spot” (140).

In another aspect, the shell (4) fits over the maxillary anterior teeth (10) and the pre-molars (190) for a total of eight teeth, all having a plurality of protrusions (80). The main difference compared to the appliance (3) shown in FIG. 2 is the configuration of the shell (4) fitting over eight teeth, i.e. anterior teeth (10) and premolars (190), and the larger number of protrusions (80) on each of the eight teeth. In this embodiment, there are two rows of three protrusions (80), and one row of two protrusions (80) on the central incisors (90) for a total of eight protrusions (80) and three protrusions (80) on each of the lateral incisors (100); with two protrusions (80) on each bilateral cuspid (110) and three protrusions (80) on each bilateral pre-molar (190).

Custom fitting permits the appliance (3) to remain in place on the teeth (10) and fit snugly on the teeth (10). The shell (4) fits over the teeth. The shell (4) is between 0.5-1 mm thick. However, the shell (4) thickness can vary. The shell (4) represents an outline of the teeth (10), that is it is based upon the exact shape of the patient's teeth (10), and does not resemble a retainer for that reason. The appliance may be worn 24 hours a day or easily removed by the patient when desired. Once removed, the appliance may be reinserted onto the teeth (10). The custom fit and the thin nature of the appliance (3) minimizes interference with eating and other patient daily activities. The appliance (3) allows the patient's mouth to close completely and comfortably. The patient will be asked to wear the appliance periodically and up to 24 hours a day, including during sleeping.

In FIG. 2-4, as disclosed herein, the platform (60) is continuous with the gum at the maxillary front six teeth (10), the two central incisors (90), two lateral incisors (100), and two cuspids (110).

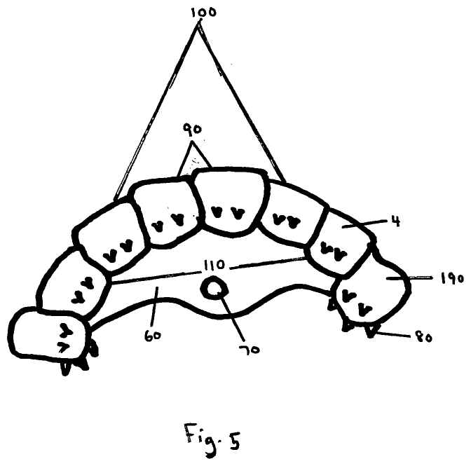

In some embodiments, the protrusions (80) are only on the maxillary front four teeth (10). FIG. 5 is a lateral embodiment, where the plurality of protrusions (80) are on the maxillary front eight teeth (10) including on the lateral side of the pre-molars (190). In several aspects, the protrusions (80) are on the lingual surface of the shell (25) covering the anterior four teeth (10) with three protrusions (80) on each of the central incisors (90) and two protrusions on each of the lateral incisors (100). In another aspect, there are no protrusions on the shell (4).

As described with respect to certain embodiments, the protrusions (80) will cause sufficient discomfort to the tip of the tongue (120) to induce the tongue (120) to withdraw away from the teeth (10). If the tongue thrust exceeds a certain level, the tongue (120) will contact the protrusions (80).

The protrusions (80) are conical in shape having a pointed end and an end perpendicular to the lingual side of the shell (4). However, they may be pin shaped, oblong or without a pointed end. The end may be circular in shape. Each of the protrusions (80) may protrude into the mouth from 1 mm to 5 mm, generally, depending on the tongue (120) response and individual patient. In one aspect, the protrusions (80) are 1 mm in length. Additionally, the protrusions (80) may be all the same size or different sizes and vary in shape. The diameter of the protrusions (80) can vary, depending on the specific patient's tongue (120) response. The protrusions are at the top portion of the tooth (10), closer to the gum line (5). The protrusions (80) may be at an angle to the lingual side of the shell (25).

The aperture (70) is located at the end of the incisor papilla (130) at the anterior part of the upper palate (50) where the tongue (120) should rest. The correct palatal position of the tongue (120) is also called the “spot” (140). The aperture (70) habituates proper lingual resting posture. The aperture (70) is configured to receive the tip of the patient's tongue (120) by acting as a tactile cue. The aperture (70) provides proprioception for where the tongue (120) tip should rest, while the tongue protrusions (80) deter the tongue (120) from resting on the teeth (10). The patient does not need to find the “spot” (140) with the tongue (120) as this will naturally happen with the appliance (3) in the mouth. The aperture (70) maximizes the space in the airway by keeping the tongue (120) from encroaching the airway space and positioning the tongue (120) in the correct resting posture up in the palate (50). The aperture supports the tongue's ability to rest in the hard palate (50) which allows the hard palate (50) to shape to the width of the tongue (120), supporting proper palatal shape and arch. This supports neuromuscular re-education for proper lingual resting posture.

In one aspect, the aperture (70) may be round and 3-5 mm in diameter. However, the aperture (70) may be oval or elliptical in shape.

The appliance (3) corrects and habituates proper lingual resting posture which builds strength in the posterior portion of the oral cavity. This improves overall resonance quality such as hyponasal/hypernasal speech quality. The appliance supports an open airway for individuals that suffer from sleep disordered breathing. The hard palate (50) is allowed to mold to the proper shape and dimension by forming to the size and shape of the tongue (120). The effect is elimination of a narrow or “V” shaped arch, maximization of the space in the nasal cavity for breathing purposes and for resonance of sounds. The appliance (3) improves overall intelligibility by improving overall resonance, articulatory precision of most lingual alveolar and lingual palatal sounds. Examples of these sounds include: s, z, t, d, l, n, sc, ch, and j. The appliance (3) improves the ability to manage saliva as a means to decreasing the “slushy sound” while articulating.

In some aspects, the appliance (3) may be configured to have a textured surface on the lingual side of the shell (25) covering the molars (200), and particularly the posterior molars (200). The textured surface provides a tactile cue for where the lateral margins of the tongue (120) should be placed during the production of the /r/ phoneme.

The appliance (3) can include lateral protrusions which are placed bilaterally or unilaterally on the lateral teeth for those users who thrust their tongue laterally (rather than anteriorly). Lateral protrusions deter the tongue from thrusting on the lingual surface of the lateral teeth.

The appliance (3) acts as a deterrent to eliminate any non-nutritive sucking habit. The protrusions (80) prevent the tongue from pushing against the teeth when swallowing and eliminates the tongue's ability to sit against the teeth at rest. The protrusions (80) act as a tactile cue and deterrent and interferes with the release of neurotransmitters, thus making the noxious habit unproductive. The appliance (3) eliminates the underside of the tongue's ability to thrust against the teeth when swallowing. The appliance (3) eliminates the tongue's ability to thrust against the teeth when speaking. The appliance (3) limits the opportunity of the tongue (120) to sit in any place created by a malocclusion. It deters sucking habits such as thumb, finger, and/or tongue sucking as well as nail biting and teeth grinding.

Mouth breathing in adults is associated with sleep disorder breathing, including snoring and obstructive sleep apnea. The appliance (3) improves nasal breathing by maximizing and conditioning breathing through the nose. It supports the habituation of a closed mouth posture for nasal breathing and supports proper oral resting posture to encourage nasal breathing. It helps to filter air through the nasal cavity and limit the impact of at least one contributing cause of hypertrophic tonsils and adenoids. It prevents alteration of the shape of the face.

The appliance (3) limits the buildup of plaque, gingivitis, gum recession and cavities.

In addition to speech-related uses, the appliance (3) has several orthodontic uses. Poor oral posture, bad oral habits, such as thumb sucking, tongue thrusting and non-nutritive sucking in a child with growing skelature, can cause problems such as malocclusion, crowding of the teeth, an open bite and narrow arch formations. The appliance (3) eliminates the tongue's ability to sit against the teeth at rest. It removes the opportunity of the tongue to sit in the space where a tooth is missing thus allowing the permanent dentition to come down in a timely manner. The appliance (3) improves alignment and occlusion, maximizes post orthodontic retention and limits regression. The appliance (3) eliminates the tongue tip's (and underside of the tongue) ability to thrust against the teeth when swallowing and eliminates the tongue tip's ability to thrust against the teeth when speaking.

The appliance (3) supports overall occlusion and retention by eliminating the tongue's ability to sit against the teeth at rest. It trains the tongue tip where to rest by placing the tongue tip on the tactile cue, the “spot”. It encourages the blade of the tongue to contact-the palate by supporting the tongue tip resting posture. It supports keeping the tongue off of the teeth at rest. That is, it supports the appropriate tongue resting posture to eliminate the tongue's pressure against the dentition. The appliance (3) supports the improvement of dental alignment/occlusion, maximizes post orthodontic retention and limits regression by keeping the tongue in the proper position at rest.

The appliance (3) reduces overall symptoms associated with temporomandibular joint disorder problems. By establishing proper oral resting posture the patient may experience reduction in teeth grinding and reduction in teeth clenching. The appliance (3) reduces the incidence of “broken teeth.”

The appliance (3) supports restful sleep. Proper tongue resting position optimizes the airway, which supports more restful sleep. Improper, low resting position of the tongue during sleep can cause the airway to narrow which can then result in sleep disordered breathing. The appliance (3) allows for proper labial closure at rest and while swallowing.

The appliance (3) may be used in both children four years of age and older and adults. The appliance (3) may be used in patients that are in the process of losing their front 4-6 baby teeth.

The appliance (3) is fitted to the exact shape of the patient's teeth and bite. The manufacture of the appliance (3) involves direct 3-D-printing which forms one part or a single piece. The process for custom fitting the appliance involves using a scanned image of the patient's mouth. An intraoral scanner, such as 3shape, takes digital images of the patient's upper teeth, lower teeth and bite. An STL file is created. The STL file is exported into Meshmixer, a 3-D CAD program, to design the specific embodiment of the appliance that the patient requires. The process is not limited to Meshmixer software and it is understood that any software program may be used. The teeth that the appliance will fit over must be selected. The area is smoothed and then inverted to delete any unwanted areas. Then an offset is used to select away from the teeth to compensate for any small errors while printing. A 0.05 offset distance is used so that the appliance will fit snugly on the patient's teeth. The offset must be created a second time to select the thickness for each appliance which ranges from 0.5 to 1 mm. The aperture (70) is then created at the end of the incisor papilla (130) which the tongue (120) should rest. The protrusions (80) are added depending on the patient by using an edit tool feature. Once the appliance is made, it must be exported again to an STL file which is then uploaded to software. Any suitable software may be used such as SprintRays cloud software or desktop software. The scanner is then configured to print based on the material used. The STL file is imported into this software program which makes supports to attach to the build plate of the 3-D scanner. The SprintRay 3-D printer prints the appliance (3) at a wavelength of between 385-405 nm. It is understood that any 3-D printer such as DLP, LCD or LED printers are suitable. The SprintRay machine uses a digital light process to cure the resin material used in a resin tank one layer at a time which is 100 microns. Once the printing is completed, a metal scraper is used to remove each appliance (3) from the build plate. The appliance (3) is then washed in an alcohol solution to remove any residual resin material. Once cleaned and dried, the appliance (3) is placed in a curing box to harden based on the specifications provided by SprintRay for the particular resin material used. The curing box is used at a wavelength between 250-390 nm. The supports are removed manually with a snipper/scissor, followed by use of a Dremel to smooth and polish each appliance (3). The appliance (3) is then disinfected and run through water to remove any polishing debris. Once dried, it is packaged.

The appliance (3) is suitably manufactured from a soft, flexible and/or resilient material, suitably a resin material including, but not limited to, material from acrylic monomers and oligomers. The material is transparent and stain-resistant. Any biocompatible (ISO 10993-1 and/or USP Class VI) 3-D printing material that has been specifically formulated for the dental industry may be used. In one example, Keysplint™ soft resin material is used. This resin has Flexural Strength (ISO 20795-2) 2.6-4.4 M, Flexural Modulus (ASTM D790) 1356, Tensile Strength (ASTM D638) 52, Tensile Young's Modulus (ASTM D638) 1790, Water Sorption (ISO 20795-2)<18 μg/mm³, Elongation at Break (ASTM D638) 110%. It should be understood that any biocompatible flexible light-curing photopolymer resin for use in 3-D printers may be used. However, the resin should be clear when cured for esthetic purposes.

The appliance (3) may be made from any plastic material capable of being molded in one piece using materials such as biocompatible thermoplastics and using processes such as injection molding. Again, the plastic should be clear for esthetic purposes.

In some embodiments, an at-home impression kit may be used for the custom fitting. The kit includes impression material with a specific set time. While standard impression putty is used, an example of which is Defend Regular Set, Base & Catalyst Putty, any impression material or combination of material may be used to obtain an impression of the patient's teeth. The impression creates a tooth alignment which can then be scanned either manually or via a desktop scanning machine.

In some embodiments, the appliance (3) can be used to simultaneously correct teeth positioning. An upper and lower shell to both the maxillary and mandibular teeth may be used to correct teeth positioning.

In some embodiments, the appliance (3) may have a microprocessor, Bluetooth or cloud, micro-SD card, sensors such as airflow sensor, pressure sensor, temperature sensor, sound sensor, data handling, and data transmission, for controlling actions of the patient.

In one example, the appliance can comprise part of a kit, where the kit comprises the appliance (3), a patient instruction booklet, and a box. In some aspects, the kit may include a myofunctional therapy training program or be combined with orthodontic devices. literature or aids.

It is understood that the present subject matter may be embodied in many different forms and should not be construed as being limited to the embodiments set forth herein. Rather, these embodiments are provided so that this subject matter will be thorough and complete and will convey the disclosure to those skilled in the art. Indeed, the subject matter is intended to cover alternatives, modifications, and equivalents of these embodiments, which are included within the scope and spirit of the subject matter as defined by the appended claims and their equivalents. Furthermore, in the detailed description of the present subject matter, numerous specific details are set forth in order to provide a thorough understanding of the present subject matter. However, it will be clear to those of ordinary skill in the art that the present subject matter may be practiced without such specific details.

The terminology used herein is for the purpose of describing particular aspects only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A tongue training appliance which is custom fitted to a patient's maxillary teeth having:
a shell which fits securely over the maxillary teeth having an anterior labial side and a posterior lingual side;
a plurality of protrusions on the posterior lingual side of the shell;
a platform extending from the posterior lingual side of the shell; and
an aperture located at an end of central incisor papilla at an anterior part of an upper palate where the tongue should rest.

2. The tongue training appliance of claim 1, wherein the shell fits over all the maxillary teeth.

3. The tongue training appliance of claim 1, wherein the shell fits over the front eight maxillary teeth and the plurality of protrusions further comprises:
eight protrusions on each of a central incisor;
three protrusions on each of a lateral incisor;
two protrusions on each of a bilateral cuspid;
and three protrusions on each of a bilateral pre-molar.

4. The tongue training appliance of claim 1, wherein the shell fits over anterior teeth.

5. The tongue training appliance of claim 2, wherein occlusal surfaces of molars are exposed and the anterior labial side of anterior teeth is exposed.

6. The tongue training appliance of claim 5, wherein the plurality of protrusions are optional.

7. The tongue training appliance of claim 1, wherein the protrusions are conical spikes extending perpendicular to central and lateral incisors.

8. The tongue training appliance of claim 7, where the protrusions protrude from 1 mm to 5 mm into a patient's mouth.

9. The tongue training appliance of claim 8, where the protrusions protrude 1 mm into a patient's mouth.

10. A tongue training appliance which is custom fitted to the patient's maxillary teeth having:
a shell which fits securely over the maxillary teeth having an anterior labial side and a posterior lingual side;
a platform extending from the posterior lingual side of the shell; and
an aperture located at the end of central incisor papilla at the anterior part of an upper palate where the tongue should rest.

11. The tongue training appliance of claim 10, further comprising protrusions, wherein the shell fits over all the maxillary teeth, occlusal surfaces of molars are exposed and an anterior labial side of anterior teeth is exposed.

12. The tongue training appliance of claim 1 wherein for patients with a lateral tongue thrust, the plurality of protrusions are placed bilaterally or unilaterally on teeth.

13. The tongue training appliance of claim 10, wherein the shell fits over all the maxillary teeth.

14. The tongue training appliance of claim 1, wherein the aperture is round and between 3 to 5 mm in diameter.

15. The tongue training appliance of claim 1 further comprising a soft resin material and prepared using a 3-D printing machine.

16. The tongue training appliance of claim 1, wherein the shell has a thickness of between 0.5 to 1 mm.

17. A method for training a tongue comprising:
providing the tongue training appliance of claim 1 causing a patient to wear the appliance;
where the aperture on the appliance is configured to provide a spot which habituates proper lingual resting posture and wherein the aperture is configured to receive a tip of the patient's tongue and acts as a tactile cue for where the tongue tip should rest, and wherein the tongue protrusions deter the tongue from resting on the maxillary teeth.

18. The method of claim 17, wherein the appliance improves overall speech resonance quality and intelligibility, decreases a “slushy sound” while articulating, improves articulatory precision of most lingual alveolar and lingual palatal sounds, wherein the sounds are s, z, t, d, l, n, sc, ch, and j.

19. The method of claim 17, wherein the appliance is useful to treat orthodontic conditions, temporomandibular joint problems, sleep apnea, snoring, noxious oral habits, and mouth breathing.

20. The tongue training appliance of claim 1 comprising a kit, wherein the kit comprises the appliance, a patient instruction booklet, and myofunctional therapy training program material.
